(19) United States
(12) Patent Application Publication
(10) Pub. No.: US 2025/0090433 A1
(43) Pub. Date: Mar. 20, 2025

(54) ORAL POUCHED COMPOSITION FOR ORAL CARE

(71) Applicant: Inside Innovation Scandinavia AB, Stockholm (SE)

(72) Inventors: Björn W. ERICSSON, Stockholm (SE); Håkan ENGQVIST, Uppsala (SE)

(73) Assignee: Inside Innovation Scandinavia AB, Stockholm (SE)

(21) Appl. No.: 18/729,237

(22) PCT Filed: Jan. 2, 2023
(86) PCT No.: PCT/SE2023/050001

§ 371 Date: Jul. 16, 2024

(30) Foreign Application Priority Data
Jan. 19, 2022 (SE) 2250045-8

(51) Int. Cl. A61K 8/24; A24B 13/00; A24B 15/16; A61K 8/02; A61K 8/73; A61K 9/00; A61Q 11/00

(52) U.S. Cl. CPC A61K 8/24; A24B 13/00; A24B 15/16; A61K 8/0204; A61K 8/731; A61K 9/0063; A61K 9/009; A61Q 11/00; A61K 2800/30; A61K 2800/92

(57) ABSTRACT

A tobacco and nicotine-free oral pouched product for dental and oral care is provided. The oral pouched product includes a saliva-permeable pouch enclosing an oral formulation including tooth re-mineralizing agent. Also described is a tobacco and nicotine-free oral pouched product for use as a medicament.

### FIELD OF INVENTION

The present invention generally relates to a composition for oral care, preferably dental care, and in particular to such a composition in the form of a pouched product.

### BACKGROUND

Dental care and oral hygiene are important aspects of personal care for many consumers. People use a variety of different products in order to maintain their dental and oral hygiene. The products include toothbrushes, toothpaste, mouth wash, etc. These products have in common that they reduce the risk for cavities, caries, bad breath, etc.

Smokeless oral tobacco products, such as snus (dry and wet) or snuff, are increasingly popular by consumers. They are used in the oral cavity in direct contact with the oral mucosa. Although it is generally considered that smokeless oral tobacco products are less harmful to the consumers than cigarettes, they still may have a negative effect, in particular on the dental and oral health.

EP 2405942B1 discloses an oral delivery product in the form of an oral pouch.

WO 2011/102788 discloses oral smokeless tobacco products and oral smokeless non-tobacco snuff products comprising carbamide or carbamide salts.

U.S. Pat. No. 9,554,986 B2 discloses an oral care composition, comprising one or more functional agents selected from a group consisting of a whitening agent, a re-mineralizing agent, an anti-plaque agent, an anti-gingivitis agent, a detoxifying agent and its combinations. The composition further comprises a probiotic blend consisting of beneficial oral bacterial population and at least one essential oil comprising antimicrobial activity.

There is still a need for an oral care product directed towards consumers of smokeless oral tobacco products and that improves the general health of the mouth cavity and/or the dental health of the consumer.

### SUMMARY

It is a general object to provide an oral pouched product that is tobacco- and nicotine-free by which the above-mentioned problems may be overcome.

It is a further object of the invention to provide a product for oral delivery of active agents, in particular an oral delivery product that can be used as a snus or snuff product.

These and other objectives are met by the embodiments as disclosed herein.

The oral pouched product is defined by claim 1.

According to one aspect of the invention there is a tobacco and nicotine-free oral pouched product, comprising a saliva-permeable pouch enclosing an oral formulation. The oral formulation comprises a tooth re-mineralizing agent.

According to one embodiment of the invention the tooth re-mineralizing agent is selected from the group consisting of a calcium silicate, a calcium phosphate, a calcium sodium phosphosilicate and any combination thereof. In a particular embodiment of the invention, the tooth mineralizing agent is β-tricalcium phosphate.

In one embodiment of the invention the amount of tooth re-mineralizing agent in the oral formulation is selected within an interval of from 0.01 to 40 wt %, preferably within an interval of from 0.1 to 20 wt % based on the total weight of the oral formulation enclosed in the saliva-permeable pouch.

According to one embodiment of the invention the oral formulation further comprises an anti-plaque agent. In one embodiment the anti-plaque agent is selected from the group consisting of pyrophosphate, zinc citrate, zinc chloride, chlorohexidine, an etheric oil, tin(II) fluoride, triclosan and any combination thereof. In one embodiment of the invention the amount of anti-plaque agent in the oral formulation is selected within an interval of from 0.01 to 10 wt % based on the total weight of the oral formulation enclosed in the saliva-permeable pouch.

According to one embodiment of the invention the oral formulation further comprises an oral mucosa improving agent. In one embodiment the oral mucosa improving agent is selected from the group consisting of a vitamin, a zinc salt, a silicon salt, and any combination thereof. In one embodiment the oral mucosa improving agent is a zinc or silicon salt, and the amount of oral mucosa improving agent in the oral formulation is selected within an interval of from 0.01 to 10 wt % based on the total weight of the oral formulation enclosed in the saliva-permeable pouch. In one embodiment of the invention the oral mucosa improving agent is at least one vitamin, and the amount of oral mucosa improving agent in the oral formulation is selected within an interval of from 0.01 to 2 wt % based on the total weight of the oral formulation enclosed in the saliva-permeable pouch.

According to one embodiment of the invention the oral formulation further comprises cellulose, preferably microcrystalline cellulose.

According to one embodiment of the invention the oral formulation further comprises a sweetener and/or a flavouring agent.

According to one embodiment of the invention the oral formulation comprises a sweetener selected from the group consisting of xylitol, sorbitol, aspartame, acesulfame K, and any combination thereof.

According to one embodiment of the invention the oral formulation comprises a flavouring agent selected from the group consisting of methanol, peppermint oil, and any combination thereof.

In a second aspect of the invention there is a tobacco and nicotine-free oral pouched product for use as a medicament.

Further aspects of the invention relate to a tobacco and nicotine-free oral pouched product (for use in treatment of oral infections, preferably caries, and/or inflammation in oral mucosa.

In the following, the invention will be described in more detail, by way of example only, with regard to non-limiting embodiments thereof.

### BRIEF DESCRIPTION OF DRAWINGS

The embodiments, together with further objects and advantages thereof, may best be understood by making reference to the following description taken together with the accompanying drawings, in which:

FIG. 1 illustrates an embodiment of an oral pouched product as seen from above (1A) and from the side (1B).

### DEFINITIONS AND ABBREVATIONS

‘oral use’—is used herein as a description for use in the oral cavity of a human, such as buccal placement;
‘oral pouched product’ or ‘pouched product’—refers herein to a composition packed in a saliva-permeable pouch material intended for oral use;
‘active agent(s)’ or ‘active ingredient(s)’—refers to a substance or ingredient that is biologically active;
‘bioglass’—refer to calcium sodium phosphosilicate, typically with the composition 45 wt % SiO₂, 24.5 wt % CaO, 24.5 wt % Na₂O and 6.0 wt % P₂O₅;
β-TCP—abbreviation for β-tricalcium phosphate, chemical formula Ca₃(PO₄)₂; and
wt %—refers to weight percent of the ingredient in relation to the total weight of the dry ingredients, i.e., excluding any moisture content.

### DETAILED DESCRIPTION

Smokeless oral tobacco products, such as snus or snuff, are becoming increasingly popular. When quitting smoking it is common to exchange cigarettes for snus, and it is expected that the number of snus consumers will increase in the coming years. With the increase in snus consumers there has also been an increase in new products like tobacco-free snus for example.

Snus is a pouched product that is typically used by the consumers by placing the pouch between the upper or lower gum and the lip and retaining it there for a limited period of time. The pouch material holds tobacco in place while allowing salvia to pass into the interior of the pouch so that flavours and nicotine are extracted from the product.

The health effects of snus are debated but consumers could experience negative effects relating to their dental health, or general health of their oral cavity. Examples of symptoms snus consumers may experience include discoloured teeth, inflammations, gum recessions, oral lesions, dry mouth, bad breath, etc. Therefore, there is a need for an oral and dental care product suitable for snus consumers.

A first aspect of the invention relates to a tobacco and nicotine-free oral pouched product, comprising a saliva-permeable pouch enclosing an oral formulation. The oral formulation comprises a tooth re-mineralizing agent.

‘Tobacco- and nicotine-free’ means that the product is essentially free from both tobacco and nicotine, however it may still comprise trace amounts of tobacco and/or nicotine. In a particular embodiment, the oral delivery product and the oral formulation is totally free from both tobacco and nicotine, i.e., does not comprise any detectable amounts of tobacco and nicotine.

The oral formulation, also referred to as oral composition, may be referred to as an ‘oral care formulation’ or ‘oral care composition’. It may additionally be referred to as an oral delivery formulation of active agents in form of an oral pouch.

Preferably, the oral formulation comprises particles, or is in the form of particles.

During use of the oral pouched product, it is placed in the oral cavity, typically between the upper or lower gum and the lip. When in position in the oral cavity, the oral pouched product is being subject to the moisture naturally present in the mouth. The moisture causes gradual dissolution of the tooth re-mineralizing agent and possible other active agents and/or additives in the oral formulation so that they are released in the oral cavity. When released the agent(s) may achieve a local biological, including therapeutic, effect in the oral cavity.

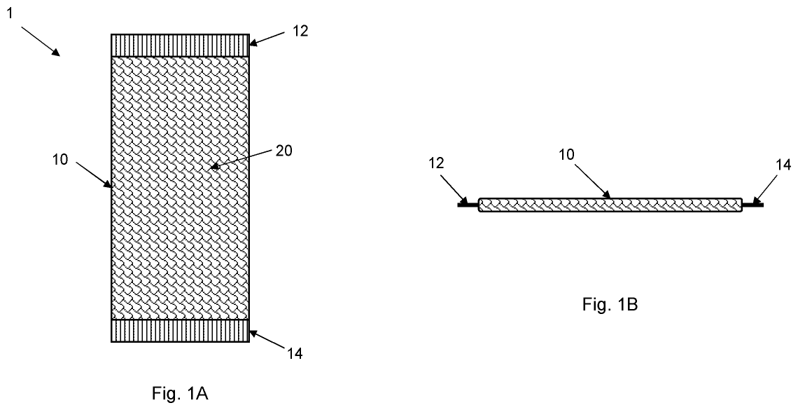

A product according to the invention is designed to generally be used in a similar way as a traditional snus or snuff product. Hence, the oral formulation delivers or administrates its active agent(s) by being placed in the oral cavity. The salvia-permeable pouch therefore preferably has a similar overall size and design for allowing it to be effectively placed in the oral cavity, preferably under the upper lip, between the upper lip and the upper gum, or under the lower lip, between the lower lip and the lower gum. The actual dimension of the pouched product can be determined by the person skilled in the art. The pouched product preferably has similar shape and size as a regular snus product present on the market today. As such it will be familiar to snus consumers and easy to use. Typically, such a product has a rectangular shape with a maximum length and a maximum width. The maximum length of the product may be between 10-30 mm, such as between 20-30 mm, and the maximum width may be between 8-15 mm, such as between 10-15 mm. The weight of the product is typically less than 1 g, such as 0.5-1.0 g. Also smaller pouch lengths and/or widths could be used, and/or smaller weights of the oral formulation enclosed in the couch, such as less than 0.5 g, for instance between 0.1 and 0.5 g. FIGS. 1A and 1B schematically illustrate an embodiment of a tobacco and nicotine-free oral pouched product 1 according to an embodiment. The oral pouched product 1 comprises the saliva-permeable pouch 10 comprising and enclosing the oral formulation 20.

The pouch 10 of the oral pouched product 1 may be any suitable saliva-permeable and preferably non-dissolvable material. Examples of materials suitable to use for a pouch include cellulose (non-woven or woven), viscose, or synthetic or natural polymers. The pouch 10 is typically sealed using a seam or a seal 12, 14, applied using for example sewing, adhesives or welding. The pouch material, i.e., the enclosed oral formulation 20, is preferably not dissolvable or at least difficult to dissolve in the oral cavity. Thus, after administration of the active agent(s) the pouch 10 with any remaining material is removed from the oral cavity and discarded.

The openings, pores, and/or channels, of the salvia-permeable pouch are preferably selected so that the particle size of the oral formulation is larger than the pore size, at least initially before being placed in the oral cavity. In this way the oral formulation generally cannot pass through the pouch, at least not during storage of the oral pouch product.

A tooth re-mineralizing agent is a compound or a mixture of compounds that has ability to release calcium and/or phosphate ions that are deposited on the tooth surface producing new mineral. In one embodiment the tooth re-mineralizing agent is selected from the group consisting of calcium silicates, preferably CaOSiO₂, calcium phosphates, preferably β-TCP, calcium sodium phosphosilicate or bioglass, and any combinations thereof. In one embodiment the amount of tooth re-mineralizing agent in the oral formulation is selected within an interval of from 0.01 to 40 wt %, preferably selected within an interval of from 0.1 to 20 wt %, based on the total weight of the oral formulation enclosed in the saliva-permeable pouch. A tooth re-mineralizing agent may re-mineralize cavities, cracks and tooth enamel. A tooth re-mineralizing agent that comprises or consist of calcium silicate(s) and/or calcium phosphates have the ability to release calcium and/or phosphate ions inside the oral cavity. When such ions are deposited on the tooth surface it can produce new tooth mineral. In this way the tooth remineralizing agents may re-mineralize cavities, cracks and tooth enamel.

In one embodiment of the invention the oral formulation further comprises an anti-plaque agent. Dental plaque is a film of bacteria, or a biofilm, present on the surface of the teeth. Dental plaque can cause dental decay, i.e., cavities or caries, and gum disease if not removed. It can also result in the formation of dental calculus. The anti-plaque agent may also be referred to as an anti-bacterial or anti-microbial agent. Anti-plaque agents include both de-sensitising agents and anti-calculus agents. In one embodiment of the invention, the anti-plaque agent is selected from the group consisting of pyrophosphate, zinc citrate, zinc chloride, chlorohexidine, an etheric oil, tin(II) fluoride, triclosan and any combination thereof. In one embodiment the amount of anti-plaque agent in the oral formulation is selected within an interval of from 0.01 to 10 wt % based on the total weight of the oral formulation enclosed in the saliva-permeable pouch.

As mentioned above, the colour of the teeth may become darker by the consumption of snus and other oral tobacco-products. In order to improve both the dental health and the physical appearance of the teeth, the oral composition may further comprise a whitening agent. A whitening agent may comprise abrasives, cleansing agents and dental bleach. A whitening agent may, for example, comprise sodium bicarbonate, calcium peroxide, sodium phosphate, or any combination thereof.

The oral mucosa is the mucosa membrane inside the mouth. The general health of the oral mucosa is important in terms of gum disease, bad breath, dental decay, etc. In one embodiment, the oral composition further comprises an oral mucosa improving agent. The oral mucosa improving agent may be selected from the group consisting of a vitamin, a zinc salt, a silicon salt, e.g. Ca-silicate, and any combination thereof. The amount of zinc or silicon salt in the oral composition may be selected within an interval of from 0.01 to 10 wt % based on the total weight of the oral formulation enclosed in the saliva-permeable pouch. The amount of vitamin in the oral composition may be selected within an interval of from 0.01 to 2 wt % based on the total weight of the oral formulation enclosed in the saliva-permeable pouch.

The oral composition may comprise a filler material. The filler material may be a particulate material, such as polysaccharides, e.g., maltitol and mannitol, and/or cellulose, e.g., microcrystalline cellulose. The filler material particles can have an average diameter in the range from one or a few micrometers up to several millimeters, such as from 1 μm up to 1 mm, and preferably from 10 μm up to 750 μm, and more preferably from 100 μm up to 500 μm. Note that above given preferred ranges of the particle size is the average particle diameter. In practical implementations the individual filler material particles may have different diameters so that the filler material particles have a distribution of particle diameters around the average diameter.

The filler material may be used as a passive filler material or it may be used as a carrier of the active agent(s). In the latter case, the active agent(s) may be adsorbed onto the surface of the filler material. The carrier may also be porous wherein the active agents could be adsorbed on the surface of the pores.

The oral composition may further comprise sweeteners, such as sorbitol, xylitol, maltitol, isomalt, aspartame, acesulfame K, saccharin, sucrose, glucose, fructose, lactose, mannitol, etc. The amount of sweetener in the oral formulation may be 0.01-10 wt %, based on the total weight of the oral formulation enclosed in the saliva-permeable pouch. In addition to, or instead of, sweeteners the oral composition may comprise flavouring agent(s). The flavouring agents may be selected from the group consisting of methanol, peppermint oil, and any combination thereof, and the amount of flavouring agent in the oral composition may be 0.01-5 wt %, based on the total weight of the oral formulation enclosed in the saliva-permeable pouch.

Even though generally not necessary for the storage, or shelf-life, of an oral pouched product it may comprise further additives, such as binders, wetting agents, stabilizing agents, absorption enhancers, and anti-oxidants for example Na-benzoate, glycerol and/or glycerin.

An oral pouched product according to the invention may be used by snus or other oral tobacco consumers in order to improve their dental and/or oral health. In a second aspect of the invention there is a use of a tobacco and nicotine-free pouched product as a dental and/or oral care product.

Further aspects of the invention relate to a tobacco and nicotine-free pouched product as defined herein for use in prevention or treatment of dental or oral infections, such as for example caries, inflammations in particular in the oral mucosa, and/or gum disease. In one aspect of the invention there is a use of an oral pouched product as a medicament. In one aspect of the invention there is an oral pouched product for use in the treatment of caries, and/or inflammations, in particular in the oral mucosa, and/or gum disease.

The term ‘treatment’ or ‘treating’ as used herein means an approach for obtaining beneficial or desired results, including clinical results. Beneficial or desired clinical results could include, for instance, alleviation or amelioration of one or more symptoms or conditions, i.e., prevent worsening, delay or slowing of disease progression, amelioration or palliation of the disease state. ‘Treatment’ or ‘treating’ may also prolong survival as compared to expected survival if not receiving any treatment.

The term ‘preventing’ or ‘prophylaxis’ as used herein means an approach in which a risk of developing a disease is reduced or prevented, including prolonging or delaying development. For instance, a patient predisposed to develop a disease, such as due to genetic or hereditary predisposition, could benefit for using the oral pouched product of the invention to prevent, or reduce the risk of caries, and/or inflammations, in particular in the oral mucosa, and/or gum disease.

All embodiments and aspects may be combined with each other.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-18. (canceled)

19. A tobacco-free and nicotine-free oral pouched product, comprising a saliva-permeable pouch enclosing an oral formulation, wherein the oral formulation comprises a tooth re-mineralizing agent selected from the group consisting of a calcium silicate, a calcium phosphate, a calcium sodium phosphosilicate and any combination thereof.

20. The tobacco and nicotine-free oral pouched product according to claim 19, wherein the tooth mineralizing agent is β-tricalcium phosphate.

21. The tobacco and nicotine-free oral pouched product according to claim 19, wherein the amount of the tooth re-mineralizing agent in the oral formulation is selected within an interval of from 0.01 to 40 wt % based on the total weight of the oral formulation enclosed in the saliva-permeable pouch.

22. The tobacco and nicotine-free oral pouched product according to claim 19, wherein the oral formulation further comprises an antiplaque agent.

23. The tobacco and nicotine-free oral pouched product according to claim 22, wherein the anti-plaque agent is selected from the group consisting of pyrophosphate, zinc citrate, zinc chloride, chlorohexidine, an etheric oil, tin(II) fluoride, triclosan and any combination thereof.

24. The tobacco and nicotine-free oral pouched product according to claim 23, wherein the amount of anti-plaque agent in the oral formulation is selected within an interval of from 0.01 to 10 wt % based on the total weight of the oral formulation enclosed in the saliva-permeable pouch.

25. The tobacco and nicotine-free oral pouched product according to claim 19, wherein the oral formulation further comprises an oral mucosa improving agent selected from the group consisting of a vitamin, a zinc salt, a silicon salt, and any combination thereof.

26. The tobacco and nicotine-free oral pouched product according to claim 25, wherein
the oral mucosa improving agent is a zinc or silicon salt; and
the amount of oral mucosa improving agent in the oral formulation is selected within an interval of from 0.01 to 10 wt % based on the total weight of the oral formulation enclosed in the saliva-permeable pouch.

27. The tobacco and nicotine-free oral pouched product according to claim 25, wherein
the oral mucosa improving agent is a vitamin; and
the amount of oral mucosa improving agent in the oral formulation is selected within an interval of from 0.01 to 2 wt % based on the total weight of the oral formulation enclosed in the saliva-permeable pouch.

28. The tobacco and nicotine-free oral pouched product according to claim 19, wherein the oral formulation further comprises cellulose.

29. The tobacco and nicotine-free oral pouched product according to claim 19, wherein the oral formulation further comprises a sweetener and/or a flavouring agent.

30. The tobacco and nicotine-free oral pouched product according to claim 29, wherein the oral formulation comprises a sweetener selected from the group consisting of xylitol, sorbitol, aspartame, acesulfame K, and any combination thereof.

31. The tobacco and nicotine-free oral pouched product according to claim 29, wherein the oral formulation comprises a flavouring agent in the form of peppermint oil.

32. A method comprising using the tobacco and nicotine-free oral pouched product according to claim 19 as a medicament.

33. A method comprising treating oral infections with the tobacco and nicotine-free oral pouched product according to claim 19.

34. A method comprising treating inflammation in oral mucosa with the tobacco and nicotine-free oral pouched product according to claim 19.
